(19) United States
(12) Patent Application Publication
(10) Pub. No.: US 2019/0274886 A1
(43) Pub. Date: Sep. 12, 2019

(54) DEVICE, SYSTEM AND METHOD FOR TREATING A EUSTACHIAN TUBE

(71) Applicant: MEDTRONIC XOMED, INC., Jacksonville, FL (US)

(72) Inventor: Kyle A. Godwin, Costa Mesa, CA (US)

(73) Assignee: MEDTRONIC XOMED, INC., Jacksonville, FL (US)

(21) Appl. No.: 16/299,579

(22) Filed: Mar. 12, 2019

Related U.S. Application Data
(60) Provisional application No. 62/647,025, filed on Mar. 23, 2018.
(60) Provisional application No. 62/641,740, filed on Mar. 12, 2018.

(51) Int. Cl. A61F 11/00; A61M 29/02; A61M 25/10

(52) U.S. Cl. CPC A61F 11/002; A61M 29/02; A61B 2017/00787; A61F 11/004; A61M 25/10181

(57) ABSTRACT

A treatment system including a therapy device for treating soft tissue inflammation of the ear. The device includes an inflation fluid supply tube, an inner member, and a balloon. The inflation fluid supply tube has an inflation lumen extending longitudinally therethrough and an opening disposed between opposing first and second ends. The inner member is formed of a shape memory material and defines distal, proximal, and center portions. The center portion extends longitudinally within the inflation fluid supply tube. The distal portion extends distally from the first end and the proximal portion extends through the opening. A lumen of the inner member is fluidly open at and between distal and proximal ends. The balloon is disposed along the distal portion, and is fluidly coupled to the inflation lumen. A tracking device for image guided navigation can be attached to the distal portion of the inner member.

### CROSS-REFERENCE TO RELATED APPLICATIONS

This Non-Provisional patent application claims the benefit of the filing dates of U.S. Provisional Patent Application Ser. No. 62/647,025, filed Mar. 23, 2018, entitled “Therapy Delivery System,” and U.S. Provisional Patent Application Ser. No. 62/641,740, filed Mar. 12, 2018, entitled “Methods, Devices and Systems for Treating Eustachian Tube Disorders,” the entire teachings of which are incorporated herein by reference.

### BACKGROUND

The Eustachian tube, sometimes referred to as the auditory tube or the pharyngotympanic tube, is a tube that connects the tympanic cavity of the middle ear to the nasopharynx. The Eustachian tube acts as a pressure equalizing tube that extends from the lateral nasopharynx to the middle ear. At the nasopharynx, the Eustachian tube is bounded by the torus of the Eustachian tube and forms the pharyngeal opening of the Eustachian tube (also known as the pharyngeal ostium). The Eustachian tube includes a cartilaginous portion and an osseous (bone) portion. There are several muscles that affect the function of the Eustachian tube, including muscles of the soft palate (e.g., the levator veli palatini and tensor veli palatini) and muscles of the ear (e.g., tensor tympani).

Eustachian tube dysfunction (ETD) is a common problem for both children and adults. ETD can be caused by inflammation of the tissue of or near the Eustachian tube, for example. ETD may result in Eustachian tube blockage and/or cause the Eustachian tube to resist opening. When the Eustachian tube is obstructed either through anatomical or inflammatory reasons, the middle ear is not able to equalize pressure which can lead to negative pressure and fluid build-up or retraction of the tympanic membrane.

Some current methods for treating ETD require modifying the ear drum or installing a prosthesis in the Eustachian tube or surrounding tissue. Current methods can have drawbacks relating to patient discomfort and ineffectiveness. For example, implantable tubes often hold the Eustachian tube in an always-open state, which may be very distracting and uncomfortable for patients. Other techniques can include applying energy, such as radiofrequency energy, to modify the mucosa or tissue. Further, implantable tubes or surgical intervention can require general anesthesia or invasive surgical access.

In some instances, it may be desirable to dilate the Eustachian tube in a patient in order to improve the Eustachian tube's function. Dilation catheter devices can be introduced into an anatomical passageway in a patient for dilation and treatment. This may include dilation of ostia of paranasal sinuses (e.g., to treat sinusitis), dilation of the larynx, dilation of the Eustachian tube, dilation of other passageways within the ear, nose, or throat, etc. The catheter and treatment device utilized depends upon the medical procedure performed, the anatomical delivery route, and the individual patient's anatomy, among other factors.

Advancing and precisely positioning a therapy device within the Eustachian tube for treatment of ETD can be difficult. A device that is too rigid can damage the anatomy of the patient during advancement due to inflexibility, while a device that is too flexible can be difficult to advance through torturous passages. Physicians who perform procedures using these devices rely in part upon experience and the known anatomy for appropriate placement. An endoscope can permit visualizing remote internal surgical locations within a patient at targeted areas by accessing those locations through a natural body lumen during ENT surgeries.

Visibility of both the anatomy and the surgical instrument are critical during surgeries of the ear, nose, and throat (ENT), including the Eustachian tube, for example. The endoscope can be used to visualize the Eustachian tube before and during a procedure to ensure the treatment device is entering the correct orifice. Endoscopes permit remote visualization within the anatomical passageway (e.g., the ear, nose, throat, paranasal sinuses, etc.) to position a balloon dilator, for example, at desired locations while a surgical procedure is being performed. While endoscopes provide internal visualization, the precise location of the endoscope within the body may not always be clear to the physician throughout the procedure, making a navigation device useful, particularly during delicate or complex procedures. An image guided system would provide guidance of the treatment device with respect to hard tissue, or bone structures, to improve the safety and accuracy of the procedure. The accuracy and ease of placement of the treatment device can be greatly enhanced by using image guidance for the device.

An improved therapy to treat soft tissue inflammation diseases of the ear due to ETD is desirable. Improved accuracy and ease of placement of a device to treat ETD is desirable. A need exists for improved methods, systems, and devices to help treat Eustachian tube malfunction.

### SUMMARY

The inventor of the present disclosure recognized that a need exists for devices, systems and methods that address one or more of the above-mentioned problems. For example, therapies to treat soft tissue inflammation diseases of the ear due to ETD, improved accuracy and ease of placement of a device to treat ETD, and/or improved methods, systems and device to help treat Eustachian tube malfunction.

Some aspects of the present disclosure relate to a treatment system for treating soft tissue inflammation of the ear. The system includes a therapy device having an inflation fluid supply tube, an inner member, and a balloon. The inflation fluid supply tube defines an inflation lumen extending longitudinally therethrough. The inflation fluid supply tube has a first end, a second end opposite the first end, and an opening disposed between the first end and the second end. The inner member extends within the inflation fluid supply tube. The inner member comprises an elongated tubular body having a distal portion terminating at a distal end, a proximal portion terminating at a proximal end, and a center portion extending between the distal portion and the proximal portion. The center portion extends longitudinally within the inflation fluid supply tube. The distal portion extends distally from the first end. The proximal portion extends through the opening. The elongated tubular body defines a lumen. The lumen is fluidly open at and between the distal end and the proximal end. The elongated tubular body is formed of a shape memory material. The balloon is disposed along the distal portion of the elongated tubular body. An interior of the balloon is fluidly coupled to the inflation lumen.

Other aspects of the present disclosure relate to a treatment system for treating a Eustachian tube of a patient. The system comprises a therapy device including an inner member, a balloon, and an inflation fluid supply tube. The inner member includes an elongated tubular member including a distal portion terminating at a distal end, a proximal portion terminating at a proximal end, and a center portion extending between the distal portion and the proximal portion. The elongated tubular member has a lumen fluidly open at the distal end and the proximal end. The elongated tubular member is formed of a shape memory material. The balloon is disposed along the distal portion of the elongated tubular member. The inflation fluid supply tube includes a tubular sheath disposed around the center portion of the elongated tubular body. An interior lumen of the inflation fluid supply tube is sized to accommodate the elongated tubular body and defines an inflation lumen configured to inflate the balloon. The inflation fluid supply tube extends to a proximal end of the balloon. The inflation fluid supply tube includes an opening disposed between a first end and a second end of the inflation fluid supply tube. The opening is configured to provide sealed passage of the elongated tubular body from an interior to an exterior of the inflation fluid supply tube.

Other aspects of the present disclosure relate to a method of dilating a Eustachian tube of a patient. The method including advancing a therapy device at least partially within a Eustachian tube of a patient. The therapy device includes an inflation fluid supply tube, an inner member including an elongated tubular member formed of shape memory material, a central portion of the inner member extending within the inflation fluid supply tube and distal and proximal end portions extending outward from the inflation fluid supply tube, and a balloon disposed on the distal portion of the elongated tubular member and fluidly coupled to an inflation lumen of the inflation fluid supply tube. The method also including sensing a position of the distal portion of the elongated tubular member within the Eustachian tube. The balloon is positioned at a target location based on the sensed position, and then inflated to an expanded state. The balloon, in the expanded state, and applies pressure against tissue of the Eustachian tube.

### BRIEF DESCRIPTION OF THE DRAWINGS

FIG. 1 is a cross-section of a portion of the anatomy of a human ear, showing the middle ear and Eustachian tube.

FIG. 2A is a side view of a portion of a therapy device in accordance with principles of the present disclosure and useful for treating a Eustachian tube in accordance with principles of the present disclosure.

FIG. 2B is a longitudinal cross-sectional view of the therapy device of FIG. 2A.

FIG. 3A is a side view of a portion of a therapy device assembly in accordance with principles of the present disclosure and useful for treating a Eustachian tube in accordance with principles of the present disclosure

FIG. 3B is a longitudinal cross-sectional view of the therapy device assembly of FIG. 3A.

FIG. 4 illustrates an example treatment system in accordance with principles of the present disclosure and including the therapy device assembly of FIG. 3A.

### DETAILED DESCRIPTION

Surgical devices and systems embodying principles of the present disclosure can be employed in various types of surgical procedures including, but not limited to, treatment of Eustachian tube dysfunction. Image-guided dilation systems in accordance with aspects of the present disclosure can enhance the safety, accuracy, and ease of positioning of a dilating balloon during a dilation procedure by providing navigational guidance of the dilation balloon placement within the anatomy relative to critical anatomical structures, such as the bony isthmus of the Eustachian tube, for example. More particularly, placing image guidance receivers on a non-ferrous inner member of the dilation system can provide low electromagnetic interference, and thus provide improved accuracy, to the navigation system.

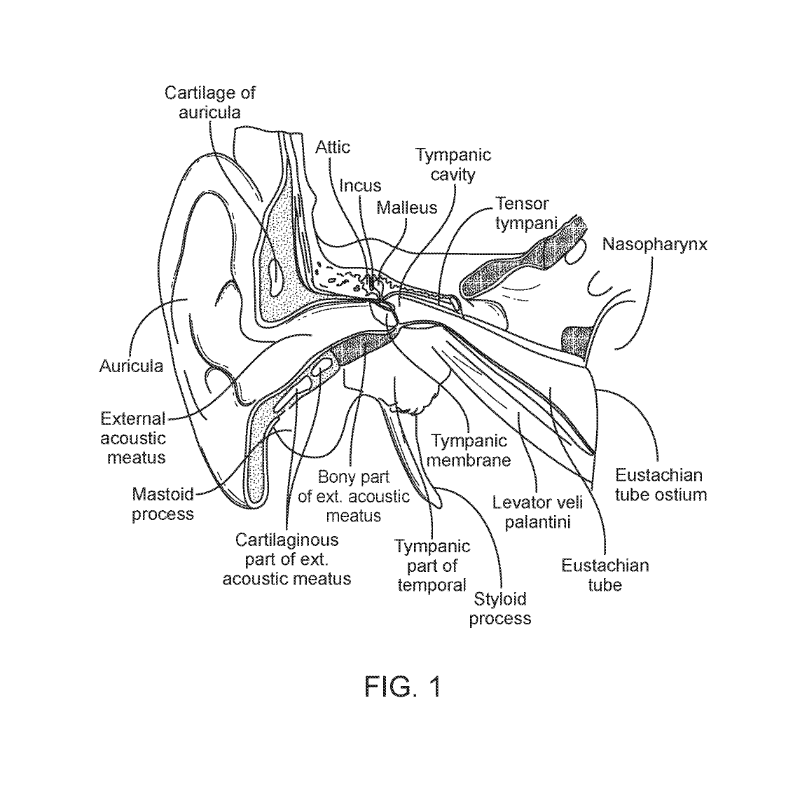

FIG. 1 illustrates an ear, including the middle ear and Eustachian tube. Some aspects in accordance with the present disclosure relate generally to a therapy delivery system for treating Eustachian tube dysfunction. The present disclosure relates more specifically to devices, systems, and methods to reduce blockage of a Eustachian tube employing the therapy delivery system. The method can include inserting a therapy delivery system transnasally or pharyngeally into the Eustachian tube. In some embodiments, therapy treatment can be applied without incisions, dissections and/or other significant trauma. Exemplary embodiments provide a therapy delivery system and method for treatment of Eustachian tube dysfunction (ETD) and allowing pressure equalization for the middle ear. Exemplary embodiments of the therapy delivery system can allow the Eustachian tube to be unobstructed at the nasopharyngeal opening through dilation of the target site.

The Eustachian tube is cartilaginous from the nasopharynx to the isthmus, at which point it is bony for the rest of the path to the middle ear. The Eustachian tube is typically approximately 20 millimeters (mm) in length. As shown in FIG. 1, the Eustachian tube is a tube that connects that tympanic cavity of the middle ear to the nasopharynx. At the nasopharynx, the Eustachian tube is bounded by the torus of the Eustachian tube and forms the pharyngeal opening of the Eustachian tube (also known as the pharyngeal ostium).

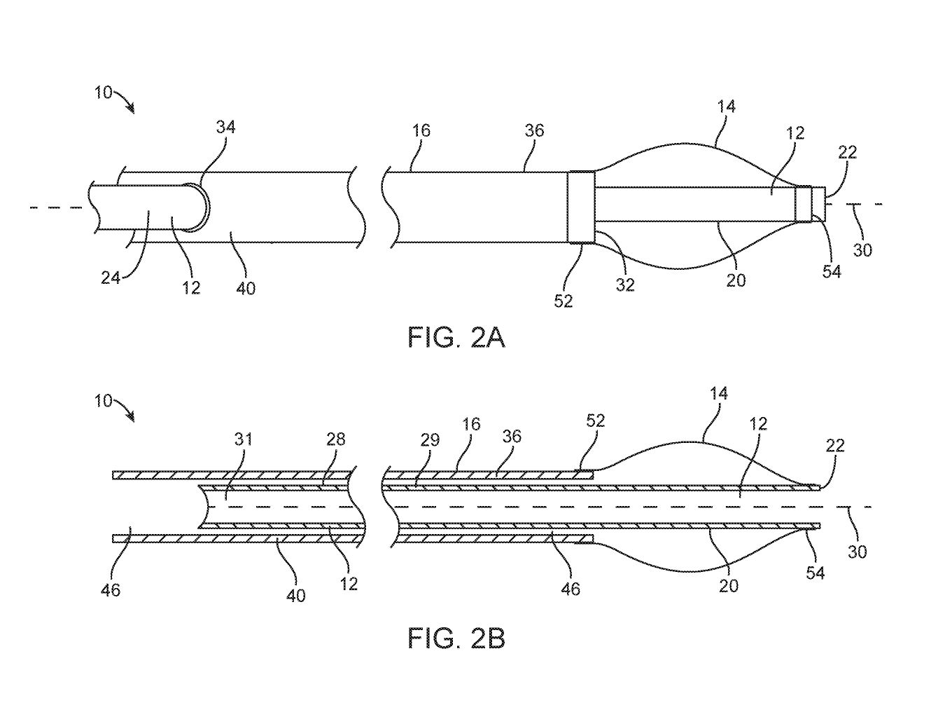

FIGS. 2A and 2B illustrate partial side and cross-sectional views of an example therapy device 10 useful for treating soft tissue inflammation diseases of the ear due to a Eustachian Tube Dysfunction (ETD) in accordance with aspects of the present disclosure. The therapy device 10 includes an inner member 12, a balloon 14, and an inflation fluid supply tube 16. Details on the various components are provided below. In general terms, however, the balloon 14 can be located and expanded at a target site within the Eustachian tube of a patient for treatment of the target site.

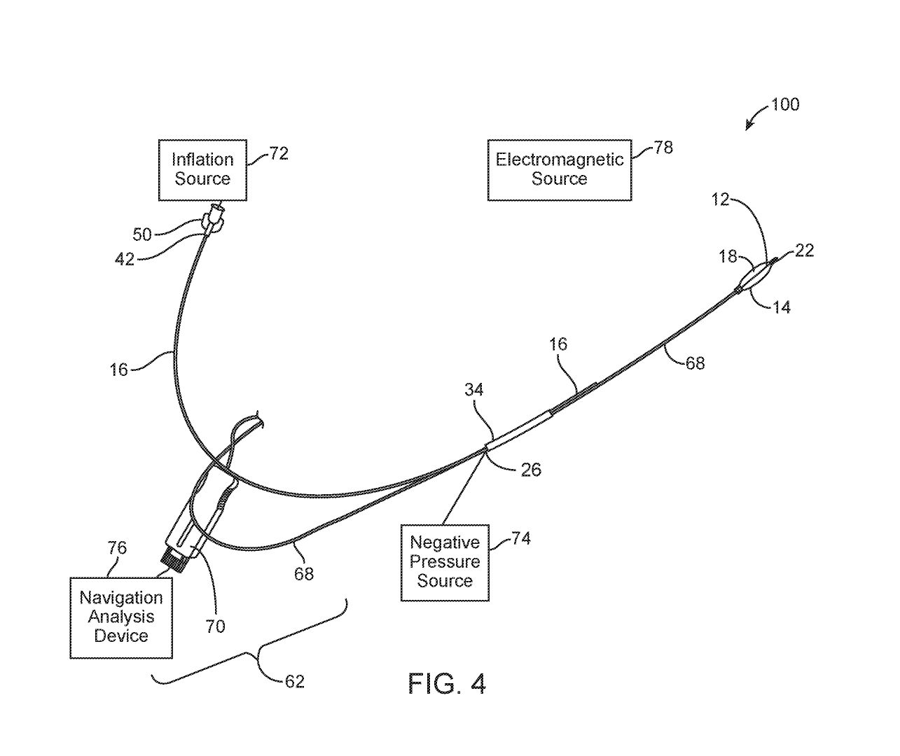

The inner member 12 includes a distal portion 20 terminating at a distal end 22, a proximal portion 24 terminating at a proximal end 26 (not shown in FIGS. 2A and 2B but identified, for example, in FIG. 4), and a center portion 28 extending between the distal portion 20 and the proximal portion 24. The inner member 12 is an elongated tubular body defining a longitudinal axis 30. The distal portion 20 can be shaped by a user or otherwise formed in a predetermined shape. The distal portion 20 can be bendable by the user without breaking or losing required strength to assume a shape useful in placement of at least the distal end 22 in the specific anatomy of a patient, and to retain substantially that shape during a dilation procedure. In some non-limiting embodiments, the distal portion 20 is shaped to include a bend that is 55°+/−10°. In one embodiment, the distal portion is 25 mm to 30 mm in length. The distal portion 20 is sized and shaped to be insertable into a patient's nostril and advancing the therapy device 10 within the Eustachian tube.

The inner member 12 includes an exterior surface 29 and an interior lumen 31. The interior lumen 31 is fluidly open through an entire length of the inner member 12 between, and including, the distal end 22 and the proximal end 26. The lumen inner member 12 can be suitable for pressure relief, irrigation, or aspiration via the lumen 31. In some non-limiting embodiments, the inner member 12 can have an outer diameter on the order of 1 millimeter (mm). Other suitable diameters are also acceptable.

The inner member 12 has a length and an outer diameter suitable to extend to and into the Eustachian tube through transnasal or pharyngeal insertion and within the inflation fluid supply tube 16. The inner member 12 should be of sufficient length to extend from the opening of the nasal passage (i.e., nostril) to the body of the Eustachian tube. The inner member 12 has a length suitable to extend fully into the Eustachian tube to position the distal portion 20 at the target site and extend the proximal portion 24 outside of a patient. The proximal end 26 can be positioned exterior of the patient to be fluidly open to atmosphere or connected to an aspiration source or irrigation source exterior the patient. In some embodiments, the proximal portion 24 can also provide for grasping by a user or robotic arm from exterior of the patient.

The inner member 12, along with other elements of the therapy delivery system 10, is formed of biocompatible materials. The inner member 12 is desirably formed of material with high electrical resistivity (i.e., low electrical conductivity). In some examples, the inner member 12 is formed of a non-ferrous material. Additionally, the material forming all or portions of the inner member 12 is malleable. The inner member 12 is formed to be manually bendable to a desired shape and to substantially retain the shape during a dilation procedure. For example, the center portion 28 and the proximal portion 24 of the inner member 12 can be malleable in conjunction with, or separate from, the distal portion 20. As used herein, the term “malleable” refers to being cable of being shaped, bent, or otherwise deformed by forces produced by manual manipulation of a human user such that the malleable element retains the deformation. The materials can be malleable, bendable, flexible and combinations of these characteristics. Portions or an entirety of the inner member 12 can be formed of nickel-titanium alloys (NiTi), nitinol, polymers and blends, and polymer composites, for example.

The inflation fluid supply tube 16 is an elongate tubular sheath sized to fit around the inner member 12 and includes or defines an inflation lumen 46 extending therethrough between opposing first and second ends 32, 42 (as a point of reference, the second end 42 is not shown in FIGS. 2A and 2B, but is identified, for example, in FIG. 4). An opening 34 is disposed between the first end 32 and the second end 42 of the inflation fluid supply tube 16. In one embodiment, the opening 34 is an aperture in a sidewall 40 of the inflation fluid supply tube 16. A distal section 36 of the inflation fluid supply tube 16, terminating at the first end 32, can be shaped by a user or otherwise formed in a predetermined shape. In one embodiment, at least the distal section 36 of the inflation fluid supply tube 16 can be formed of a malleable material. Although shown straight, the distal section 36 can be bendable by the user without breaking or losing required strength to assume a different shape useful in placement in the specific anatomy of a patient, and to retain substantially that shape during a dilation procedure. The inflation fluid supply tube 16 is formed of a material that does not substantially change in length during pressurization, such as pressurization used to inflate the balloon 14. The inflation fluid supply tube 16 is formed of a material suitably rigid to maintain the inflation lumen 46. The second end 42 of the inflation fluid supply tube 16, opposite the first end 32, can include or be coupled to a connection member 50 (see, e.g., FIG. 4).

The balloon 14 can be an expandable balloon that is transitionable between a low-profile contracted state against the inner member 12 and an expanded, inflated state. The balloon 14 is illustrated in the expanded state. The balloon 14 is sized and shaped for dilating a patient's Eustachian tube when transitioned to or toward the expanded state. The balloon 14 has a proximal end 52 and a distal end 54. In one embodiment, the balloon 14 has a length (distance between the distal and proximal ends 52, 54) of not more than 25 mm, in some embodiments in the range of 15-25 mm, and in some embodiments approximately 20 mm, and has an expanded diameter of 5 mm to 7 mm. In some embodiments, the balloon 14 can be transparent or translucent, as shown. The balloon 14 can be formed of a flexible biocompatible material. Any suitable expandable medical balloon material and construction available may be used.

When assembled, the inner member 12 can be longitudinally fixed within the inflation fluid supply tube 16. More particularly, the center portion 28 of the inner member 12 is disposed within the inflation fluid supply tube 16. The center portion 28 of the inner member 12 can extend within the inflation lumen 46 of the inflation fluid supply tube 16 or can be fluidly separated from the inflation lumen 46 (not shown). In one embodiment, the inflation fluid supply tube 16 is heat welded onto the inner member 12. In one embodiment, the inner member 12 is adhered to the inflation fluid supply tube 16 at the first end 32. In another embodiment, the inner member 12 is adhered to the inflation fluid supply tube 16 within the inflation fluid supply tube 16.

The distal portion 20 and proximal portion 24 of the inner member 12 extend and terminate outside of the inflation fluid supply tube 16. The proximal portion 24 of the inner member 12 extends through the opening 34 in the sidewall 40 of the inflation fluid supply tube 16. The opening 34 is configured to provide sealed passage of the inner member 12 from an interior to an exterior of the inflation fluid supply tube 16. The inflation fluid supply tube 16 can be fluidly sealed around the exterior surface 29 of the inner member 12 at the opening 34. For example, the opening 34 can be sealed against the inner member 12 with an interference fit, an adhesive or knurled. Other suitable means of sealing are also acceptable. The opening 34 is positioned along the inflation fluid supply tube 16 such that the proximal portion 24 of the inner member 12 a suitable length from the first end 32 so as to be located at the exterior of the patient when the distal portion 20 is inserted at the target treatment site.

The distal portion 20 of the inner member 12 extends distally through and at least partially beyond the first end 32 of the inflation fluid supply tube 16. The distal portion 20 can extend partially within the distal section 36 of the inflation fluid supply tube 16. The distal portion 20 of the inner member 12 extends beyond, or distal to, the first end 32 of the inflation fluid supply tube 16 to maintain the balloon 14. The balloon 14 is disposed around the inner member 12 with the distal end 54 of the balloon 14 coupled to the distal portion 20 of the inner member 12. The inner member 12 is suitably rigid to provide support to the inflation fluid supply tube 16 and balloon 14 yet suitably shapeable, or conformable, to extend within an insertion path of the patient without unintended damage (e.g., puncture) of the anatomy. The distal portion 20 of the inner member 12, along with the distal section 36 of the inflation fluid supply tube 16, provides suitable flexibility to the therapy delivery system 10 and can be bent to a desired shape to accommodate the patient's anatomy. In one embodiment, the inner member 12 and/or the inflation fluid supply tube 16 can be formed into a pre-determined geometry during manufacturing. In another embodiment, the inner member 12 and/or the inflation fluid supply tube 16 are formed to a determined shape by the surgeon or user.

The balloon 14 is mounted adjacent the distal end 22 of the inner member 12 along the distal portion 20 with the proximal end 52 of the balloon 14 coupled to the inflation fluid supply tube 16. The balloon 14 can be fixedly coupled to the inner member 12 and the inflation fluid supply tube 16 by welding, adhesive, or other suitable means. In one embodiment, the balloon 14 is disposed along the distal portion 20 with the distal end 54 disposed a suitable distance from the terminal distal end 22 of the inner member 12 to seal and couple the balloon 14 against the inner member 12 and maintain the internal pressure of the balloon 14 when inflated to the expanded state. In one embodiment, the distal end 54 of the balloon 14 is positioned 3 millimeters to 4 millimeters (mm) from the distal end 22 of the inner member 12. The first end 32 of the inflation fluid supply tube 16, and in particular, the inflation lumen 46, fluidly communicates with and terminates at the balloon 14 to inflate, or expand, the balloon 14, for example. The inflation fluid supply tube 16 can fluidly couple the inflation lumen 46 at the first end 32 of the inflation fluid supply tube 16 to the balloon 14 for rapid inflation and deflation of the balloon 14. The inflation fluid supply tube 16 does not expand under the pressures used to expand the balloon 14.

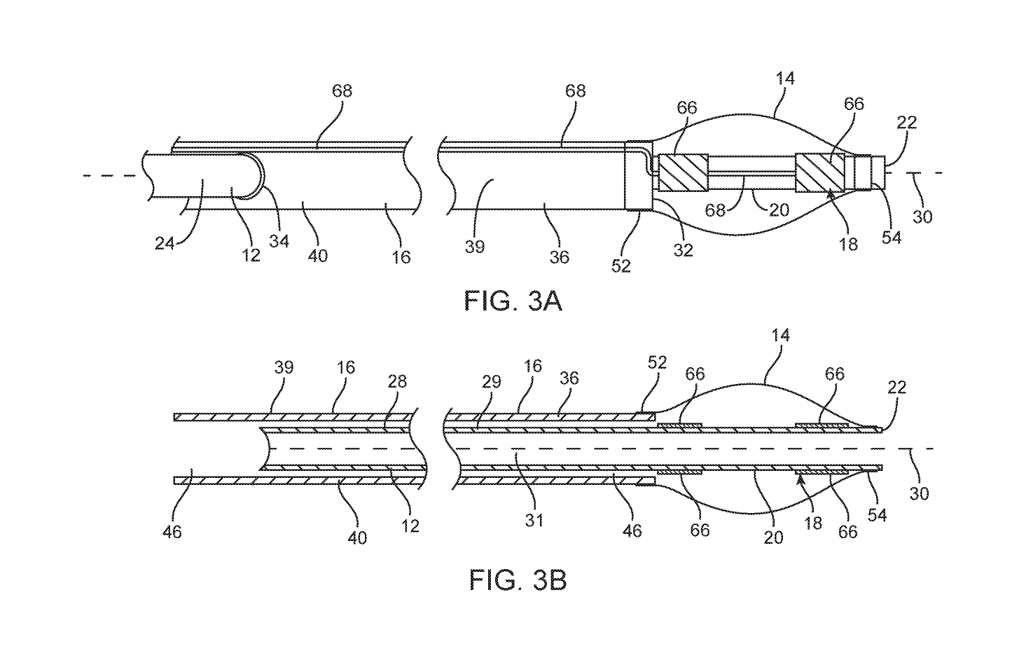

With additional reference to FIGS. 3A and 3B, a tracking device 18 can be assembled to the therapy device 10, resulting in a therapy device assembly in accordance with aspects of the present disclosure in which the tracking device 18 assists with navigation and accurate placement of the balloon 14 within a patient's anatomy. As a point of reference, and as described in greater detail below, the tracking device 18 can be interconnected to, or considered part of, an image guided navigation system 62 (FIG. 4). In general terms, the tracking device 18 is configured to receive and/or transmit electromagnetic current implicating a three-dimensional position, as discussed further below.

In some embodiments, the tracking device 18 can include one or more conductive receivers 66 and a conductor 68 electrically coupled to the receiver(s) 66. In one embodiment, the receiver 66 is formed of or as a coiled wire wound about a portion of the therapy device 10 (e.g., a ferromagnetic wire wound around the inner member 12 with a predetermined number of wrappings), a clamp or collar that extends completely or partially around a component of the therapy device 10, or any other configuration (e.g., ferromagnetic element) that can be securely attached to the therapy device 10. In one embodiment, the conductor 68 can be coated, or encased, in a biocompatible material where exposed. The conductor 68 extends from the corresponding receiver 66 to a connector 70 (shown in FIG. 4). The connector 70 can be any suitable type of electrical connector, such as a universal serial bus (USB) connector, for example, with the conductor 68 transferring electrical signals between the connector 70 and the corresponding receiver 66. Components of the tracking device 18 are preferably flexible and thin. The choice of material for components of the tracking device 18 should consider the need for flexibility, thinness, and conductivity. For example, components of the tracking device 18 can be made from copper formed as a wire. Other suitable materials are also acceptable.

The receiver(s) 66 can be disposed along the distal portion 20 of the inner member 12. In one embodiment, the one or more receivers 66 is adhered to the inner member 12. The one or more receivers 66 can be disposed on the distal portion 20 of the inner member 12 within the balloon 14 such that it is isolated and protected from fluids or tissue when the therapy delivery system 10 is being used within a patient.

In some embodiments, the receiver 66, formed of a coiled wire, includes one or more loops extending circumferentially around the inner member 12 along the distal portion 20. By looping around the inner member 12, the receiver 66 occupies a minimum volume on the inner member 12 so that the tracking device 18 remains as thin as possible. The conductor 68 extends from the receiver 66 along the distal portion 20 of the inner member 12 and along the inflation fluid supply tube 16. In one embodiment, the conductor 68 can be disposed along an exterior surface 39 of the inflation fluid supply tube 16. In one embodiment, the conductor 68 is coupled to, or attached to, the inflation fluid supply tube 16 for a desired length. In one example, the conductor 68 is coupled to the inflation fluid supply tube 16 for a length suitable to extend together to an exterior of a patient and then independently from thereon. In one embodiment, the conductor 68 is encased in a biocompatible material where extended along the inflation fluid supply tube 16, outside of the balloon 14, to the connector 70.

The tracking device 18 is preferably flexible so that it does not interfere with the normal functioning of the therapy device 10. The tracking device 18 may therefore be steered or advanced into position with the therapy device 10 such that the tracking device 18 is passively advanced along with the therapy device 10. In addition, the tracking device 18 is preferably thin, so that it only minimally increases the profile of the therapy device 10. In this way, the tracking device 18 does not interfere with the access through the insertion pathway that the therapy device 10 traverses.

In some embodiments, the receiver(s) 66 receives electrical currents around the inner member 12 during use. The inner member 12 formed of a non-ferrous material having relatively high electrical resistivity, such as nitinol, for example, can provide less electromagnetic interference, and thus provide increased accuracy, to the navigation system 62 than available with other materials (e.g., stainless steel, aluminum). For example, nitinol has a significantly higher electrical resistivity (approximately 30 times higher) than aluminum, resulting in lower induced currents in the inner member 12 formed of nitinol than if formed of aluminum. The higher electrical resistivity of the nitinol formed inner member 12 assists with improving efficiency of the receiver 64 disposed around the distal portion 20 of the inner member 12 and reduce positional errors in identifying the position of the receiver(s) 64, and thus, improve the accuracy of the balloon 14 placement in the anatomy.

FIG. 4 illustrates an example treatment system 100 in accordance with principles of the present disclosure, including the therapy device 10 (labeled in FIG. 2A) carrying the tracking device 18, useful for treating soft tissue inflammation of the ear, such as Eustachian tube dysfunction in accordance with aspects of the present disclosure. The treatment system 100 can include the therapy device 10 and the image guided navigation system 62 discussed above, an inflation source 72, and an optional negative pressure source 74. The connection member 50 of the inflation fluid supply tube 16 is suitable for connecting to the inflation source 72 to inflate the balloon 14. The proximal end 26 of the inner member 12 can be coupled to the negative pressure source 74, if desired. The interior lumen 31 (FIG. 2A) of the inner member 12 is suitable for pressure relief or aspirating diseased tissue or mucosa from the Eustachian tube anatomy, for example, while the balloon 14 is inflated to dilate the Eustachian tube.

The image guided navigation system 62 can include a navigation analysis device 76 electrically coupled to the tracking device 18 via the connector 70 for minimally invasive dilation treatment procedures. The navigation analysis device 76 can take a wide variety of forms and can be included in an Integrated Power Console (IPC), for example. In general terms, the navigation analysis device 76 facilitates operation of the image guided navigation system 62, processing and monitoring of the images. The navigation analysis device 76 can include image guided software. In one embodiment, an IPC including the navigation analysis device 76 can be connected to the inflation source 72 and the negative pressure source 74 for control of the sources 72, 74.

The inner member 12 can be maneuvered transnasally or pharyngeally into the Eustachian tube along an insertion path without damage to soft tissue. Use of the therapy device 10 includes inserting the distal end 22 of the inner member 12 into the passageway of the patient and advancing the therapy device 10 to the desired position or target site. If desired, a guide catheter (not shown) can be used to insert the therapy device 10, however, the inner member 12 is suitably rigid to allow insertion without use of a guide catheter in some embodiments. Attaining the desired position can be sensed, or determined, with the assistance of the image guided navigation system 62.

With continued reference to FIG. 4 and additional reference to FIGS. 3A-3B, the image guided navigation system 62 includes an electromagnetic source 78, positioned external to the patient, to establish a magnetic field that induces a voltage in the receiver(s) 66 mounted on the inner member 12 of the therapy device 10, which has otherwise been inserted within the body of the patient disposed within the magnetic field. The voltage of each receiver 66 is dependent upon the location and orientation of the respective receiver 66 within the magnetic field. By sensing and processing current conducted from each receiver 66, the navigation analysis system 76 can determine the location of each receiver 66 with respect to one another and provide a visual map to aid the operator in navigating the distal portion 20 of the therapy device 10 to a target site within the body of the patient. This navigation capability allows the clinician to identify the location of the tracking device 18, and therefore of the distal portion 20 and the balloon 14 of the therapy device 10, within the body. Image data can be obtained via the tracking device 18. The tracked location of the therapy device 10 can be displayed relative to the navigation data. Images for a variety of perspectives (e.g., axial, sagittal, coronal) can be displayed. The image guided navigation system 62 can provide real-time positioning information to assist in guiding and placement of the therapy device 10 to confirm desired placement of the balloon 14 and prevent over-insertion of the therapy device 10 into the isthmus of the middle ear. Navigationally guided treatment can utilize image data obtained prior to or during the dilation treatment procedure to assist the clinician, or surgeon, in performing and navigating a treatment procedure.

The balloon 14 can be expanded after the inner member 12 is inserted into the Eustachian tube. For example, the balloon 14 can be expanded after the inner member 14 is inserted into the ostium in order to facilitate dilating and treating. Once the inner member 12 has been desirably positioned against the tissue surface at the target site, the balloon 14 disposed on the distal portion 20 of the inner member 12 can be inflated with a fluid, such as water or saline, or a gas, such as air, delivered from the inflation source 72 through the inflation lumen 46 of the inflation fluid supply tube 16. The balloon 14 in the expanded state to bear against a wall of the Eustachian tube. The balloon 14 is disposed along the distal portion 20 of the inner member 12 to maintain the balloon 14 a suitable distance from being positioned and dilated against undesired anatomy such as bone. The balloon 14 is configured to reshape and/or deform tissue when the balloon 14 is inflated to the expanded state to press against, and apply pressure to, the tissue. For example, soft tissue having a diseased outer state can be pushed against with the balloon 14 in the expanded state to expand a passageway of the tissue treatment surface of the Eustachian tube at the target site. Once the desired treatment has been completed, the balloon 14 may be deflated, or returned to the contracted state, and the distal portion 20 of the inner member 12 may be moved to another region of the tissue or removed entirely.

The lumen 31 of the inner member 12 can provide pressure relief while dilating the Eustachian tube. In one embodiment, diseased tissue or mucosa can be suctioned out from the Eustachian tube anatomy through lumen 31 of the inner member 12. The balloon 14 can remain inflated for a determined amount of time to press against the soft tissue of Eustachian tube walls to expand the Eustachian tube opening. The pressed tissue can maintain the expanded Eustachian tube opening after the balloon 14 is collapsed and the therapy delivery device 10 is withdrawn and removed from the patient. In some embodiments, Eustachian tube opening can remain expanded for six weeks or more after receiving therapy.

Although the present disclosure has been described with reference to preferred embodiments, workers skilled in the art will recognize that changes can be made in form and detail without departing from the spirit and scope of the present disclosure

What is claimed is:

1. A treatment system for treating soft tissue inflammation of the ear, the system comprising:
a therapy device comprising:
an inflation fluid supply tube having an inflation lumen extending longitudinally therethrough, the inflation fluid supply tube having a first end, a second end opposite the first end, and an opening disposed between the first end and the second end;
an inner member extending within the inflation fluid supply tube, the inner member comprising an elongated tubular body having a distal portion terminating at a distal end, a proximal portion terminating at a proximal end, and a center portion extending between the distal portion and the proximal portion, the center portion extending longitudinally within the inflation fluid supply tube, the distal portion extending distally from the first end, the proximal portion extended through the opening, the elongated tubular body having a lumen, the lumen fluidly open at and between the distal end and the proximal end, the elongated tubular body formed of a shape memory material; and
a balloon disposed along the distal portion of the elongated tubular body, the balloon fluidly coupled to the inflation lumen.

2. The system of claim 1, further comprising:
an image guided navigation system including a tracking device attached to the elongated tubular body.

3. The system of claim 2, wherein the tracking device includes a conductive coil secured to the distal portion of the elongated tubular body.

4. The system of claim 2, wherein the tracking device is configured to transmit electromagnetic current indicative of a three-dimensional position.

5. The system of claim 1, wherein the elongated tubular body has a diameter from 1 millimeter to 2 millimeters.

6. The system of claim 1, wherein a distal end of the balloon is positioned 3 millimeters to 4 millimeters from the distal end of the elongated tubular member.

7. The system of claim 1, wherein the shape memory material is a nitinol shape memory material.

8. A treatment system for treating a Eustachian tube of a patient, the system comprising:
a therapy device comprising:
an inner member comprising an elongated tubular member including a distal portion terminating at a distal end, a proximal portion terminating at a proximal end, and a center portion extending between the distal portion and the proximal portion, the elongated tubular member having a lumen fluidly open at the distal end and the proximal end, the elongated tubular member formed of a shape memory material;
a balloon disposed along the distal portion of the elongated tubular member; and
an inflation fluid supply tube comprising a tubular sheath disposed around the center portion of the elongated tubular body, an interior lumen of the inflation fluid supply tube sized to accommodate the elongated tubular body and defining an inflation lumen configured to inflate the balloon, the inflation fluid supply tube extending to a proximal end of the balloon, the inflation fluid supply tube including an opening disposed between a first end and a second end of the inflation fluid supply tube, the opening configured to provide sealed passage of the elongated tubular body from an interior to an exterior of the inflation fluid supply tube.

9. The system of claim 8, further comprising:
an image guiding system including a tracking device attached to the elongated tubular body.

10. The system of claim 9, wherein the tracking device includes a conductive coils adhered to the elongated tubular member within the balloon.

11. The system of claim 8, wherein the system further comprises an inflation source fluidly coupled to the inflation lumen.

12. The system of claim 8, wherein the elongated tubular member is configured to maintain a pre-determined geometry.

13. The system of claim 8, wherein the elongated tubular member is formed of nitinol.

14. The system of claim 8, wherein the balloon is not more than 25 millimeters in length.

15. A method of dilating a Eustachian tube of a patient, the method comprising:
advancing a therapy device at least partially within a Eustachian tube of a patient, the therapy device including:
an inflation fluid supply tube,
an inner member including an elongated tubular member formed of shape memory material, a central portion of the inner member extending within the inflation fluid supply tube and distal and proximal end portions extending outward from the inflation fluid supply tube, and
a balloon disposed on the distal portion of the elongated tubular member and fluidly coupled to an inflation lumen of the inflation fluid supply tube;
sensing a position of the distal portion of the elongated tubular member within the Eustachian tube;
positioning the balloon disposed at the distal portion of the elongated tubular member at a target location based on the sensed position;
inflating the balloon to an expanded state through the inflation lumen; and
applying pressure against tissue of the Eustachian tube with the balloon in the expanded state at the target location.

16. The method of claim 15, further comprising:
relieving pressure from within the Eustachian tube of the patient through an open lumen of the elongated tubular member, wherein fluid from the Eustachian tube enters a fluidly open distal end of the elongated tubular member and exits a fluidly open proximal end of the elongated tubular member.

17. The method of claim 16, wherein the step relieving pressure occurs during the step of inflating the balloon.

18. The method of claim 15, wherein the step of sensing a position includes operating an image guided navigation system.

19. The method of claim 18, wherein a tracking device of the image guided navigation system includes a receiver positioned within an interior of the balloon along the distal portion of the elongated tubular member.

20. The method of claim 15, further comprising:
maintaining the balloon in the expanded state for a predetermined time.
